(19) United States
(12) Patent Application Publication
(10) Pub. No.: US 2010/0143500 A1
(43) Pub. Date: Jun. 10, 2010

(54) Methods and Compositions for Inhibiting GSK-3 In Glial Cell Related Disorders

(75) Inventors: Sean E. Lawler, Columbus, OH (US); Michal Oskar Nowicki, Columbus, OH (US); E. Antonio Chiocca, Powell, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/447,811

(22) PCT Filed: Oct. 31, 2007
(86) PCT No.: PCT/US07/23001

§ 371 Date: Jan. 26, 2010

Related U.S. Application Data
(60) Provisional application No. 60/855,494, filed on Oct. 31, 2006.

(51) Int. Cl. A61K 33/14; A61K 31/4015; A61K 31/426; C12Q 1/48; C12N 9/12; C07D 207/24; C07D 277/38; C01D 15/04; A61P 35/00

(52) U.S. Cl. 424/677; 514/425; 514/371; 435/15; 435/194; 548/544; 548/192; 423/499.3

(57) ABSTRACT

A method of treating a glial cell related disorder in a mammalian subject includes administering a drug which enhances or prolongs GSK-3 α or β inactivation.

### CROSS-REFERENCE TO RELATED APPLICATIONS

This application claims the benefit of U.S. Provisional Application No. 60/855,494, filed Oct. 31, 2007, the disclosure of which is incorporated herein by reference.

### STATEMENT REGARDING FEDERALLY SPONSORED RESEARCH

This invention was made with Government support and the Government has rights in this invention under the grant under the National Institutes of Health Grant CA085139.

### TECHNICAL FIELD AND INDUSTRIAL APPLICABILITY OF THE INVENTION

This invention is directed to compositions and methods for treating or ameliorating a kinase mediated disorder. More particularly, this invention is directed to using GSK-3 inhibitor compounds to treat glial cell disorders such as glial tumors and to treat and/or regenerate glial nerve cells.

### BACKGROUND OF THE INVENTION

Invasiveness is one of the main hallmarks of primary brain tumors (1). Malignant cells diffusely infiltrate normal brain tissue and migrate along defined structures of the brain. This prevents complete surgical tumor removal and contributes to the continued poor prognosis (median survival 12 months) seen in glioblastoma multiforme, the most common primary brain tumor, with approximately 15,000 new patients diagnosed in the USA every year.

Glioma invasion is associated with specific molecular alterations, including changes in extracellular matrix composition, cell adhesion, and cytoskeletal dynamics. However, no anti-invasive therapeutics have yet translated to the clinic. Therefore, for the majority of patients, there is a need for the development of other forms of therapy.

It is also clear that there remains a need in the art for a therapeutic method to target tumor cells that invade normal brain for the effective treatment of infiltrating gliomas.

### SUMMARY OF THE INVENTION

In a broad aspect, there is provided herein a method of treating a glial cell related disorder in a mammalian subject comprising administering a drug to the subject, wherein the drug enhances or prolongs GSK-3 α or β inactivation. In one embodiment, the drug can block glial cell invasion or inhibit glial cell migration.

In a particular aspect, the glial cell related disorder comprises one or more gliomas. In one embodiment, the glial cell related disorder leads to glioma spheroid expansion.

In another embodiment, the subject is in need or regenerating nerve cells.

In another embodiment, the glial cell related disorder is characterized by misregulation of GSK-3.

In another embodiment, the glial cell related disorder can including a need to inhibit recurrence of a glioma tumor.

In a particular aspect, the drug can comprises one or more of: LiCl, SB415286 and AR-A-14418.

In another broad aspect, there is provided herein a method for screening or identifying a compound useful in treating glial cell related disorders, comprising identifying inhibitory and stimulatory compounds capable of enhancing or prolonging GSK-3 α or β inactivation. In certain embodiments, the method is useful as a therapeutic tool to prevent recurrence or further tumor spread.

In another broad aspect, there is provided herein a medicament for treating a glial cell related disorder in a mammalian subject where the medicament includes a drug which drug enhances or prolongs GSK-3 α or β inactivation. In certain embodiments, the drug can block glial cell invasion or inhibit glial cell migration. Also, in certain embodiments, medicament is useful where the subject is in need or regenerating nerve cells.

In yet another broad aspect, there is provided herein a biomarker for glioma cell invasion or migration that comprises one or more glycogen synthase kinase-3 (GSK-3). Also provided herein is a method for evaluating invasion or migration of glial cells which includes using glycogen synthase kinase-3 (GSK-3) as a biomarker.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1. Inhibition of glioma spheroid invasion by LiCl:

FIG. 1A shows the effects of LiCl on spheroids in collagen. U87 control spheroid after 96 hours expansion in type I collagen with 20 mM NaCl. Inset shows an identical spheroid grown in the presence of 20 mM LiCl (Bar=100 μm).

FIG. 1B shows the dose-dependence of invasion blockade. Time course of U87 spheroid invasion, in the presence of increasing concentrations of LiCl. Measurements were taken daily over a period of four days.

FIG. 1C shows the reversibility of effects of 20 mM LiCl. Time course of spheroid expansion with 20 mM LiCl washed out from U87 spheres at 24 hour intervals. Subsequent expansion was monitored microscopically.

FIG. 1D shows the effects of LiCl on a panel of glioma cell lines. Spheroids from 6 glioma cell lines were grown in the presence of LiCl (10 mM or 20 mM). The bar graph shows spheroid size as a percentage of control spheres at the 96 hour time point.

FIG. 2. Involvement of GSK-3 in glioma spheroid invasion:

FIG. 2A shows the effects of the specific GSK-3 inhibitors AR-A014418 and SB415286 on spheroid expansion. The graph shows a dose dependent decrease in spheroid expansion in U87 and X12 cells as a percentage of control spheres at 96 hours.

FIG. 2B shows the assessment of the involvement of inositol monophosphatase in spheroid expansion. U87 spheroids were grown in increasing concentrations of LiCl, in the presence or absence of 10 mM myo-inositol, or with the specific inositol monophosphatase inhibitor (L690, 330). The graph shows U87 spheroid size, as a percentage of control spheres at 96 hours.

FIG. 2C shows TCF/Lef-dependent activation of luciferase gene transcription is stimulated by GSK-3 inhibitors. U87 cells transfected with pSuper8XTOPflash were cultured with increasing concentrations of GSK-3 inhibitors, and promoter activity determined by luciferase levels.

FIG. 2D shows the effect of GSK-3 inhibition in a wound-healing assay. The graph shows the size of the gap left between migrating edges of U373 glioma cells grown as monolayers in the presence of GSK-3 inhibitors. siRNA knockdown of GSK-3α and GSK-3β also reduced cell motility in this assay. The knockdown of GSK-3 isoforms is shown as a Western blot (inset).

FIG. 3. LiCl affects glioma cell movement in brain slices and induces pronounced changes in glioma cell shape:

FIG. 3A shows migration of a U87 spheroid on a brain slice. Tumor cells are visualized by immunohistochemical anti-vimentin (green) staining. Cell nuclei stained with Hoechst 33528 are shown in red.

FIG. 3B shows U87 spheroid on a brain slice treated with LiCl for 24 hours.

FIG. 3C shows U87 spheroid invading collagen I matrix. A U87 spheroid was grown for 48 hours and stained with alexafluor-568 phalloidin. The image shows a quadrant of the spheroid (Bar=100 μm). Inset shows a detailed image of a migrating cell (Bar=10 μm).

FIG. 3D is similar to FIG. 3C, except cells were grown for 24 hours, and treated with LiCl for 24 hours, note the marked change in cell shape after treatment.

FIG. 4. Activation of GSK-3β in glioma invasion:

FIG. 4 shows immunostaining of section of X14 human glioma xenograft in mouse brain. Nuclear staining with Hoechst 33258 is shown in blue, (dense nuclei show the area of tumor) and phospho-ser⁹ GSK-3β in green staining outside the tumor.

FIG. 4B is similar to FIG. 4A, showing invasion of tumor in corpus collosum.

FIG. 4C shows the time course of GSK-3β phosphorylation during spheroid invasion in vitro. X12 cells were harvested throughout the invasion time course and blotted for the proteins as indicated.

### DETAILED DESCRIPTION OF THE PREFERRED EMBODIMENT

GSK-3 is a multi-functional serine-threonine protein kinase found in all eukaryotes that regulates diverse processes including metabolism, cell fate specification, cell division, and cell death (2, 3). GSK-3 functions in multiple pathways including Wnt, notch, receptor tyrosine kinase, and G-protein coupled receptor signaling. There are two closely related isoforms, GSK-3α and GSK-3β, which have both distinct and overlapping functions. GSK-3 is regulated by protein interactions; for example, in Wnt signaling, inactivation of GSK-3 by Wnt ligand stimulation leads to β-catenin stabilization and gene transcription. Independently of Wnt signaling, GSK-3 can be inactivated by phosphorylation at an N-terminal serine (Serine-9 in GSK-3β). This can be mediated by numerous upstream kinases including Akt, protein kinase A, and protein kinase C.

Pharmacological GSK-3 inhibition prevents epithelial cell migration (10), lamellipodia extension in keratinocytes (11), and filopodia formation in neurons (12). Conversely, GSK-3 inhibition promotes invasiveness of colon cancer cells (13). GSK-3 has multiple distinct functions in cell migration, involving both inactivation and activation of GSK-3. This suggests the existence of independent functions in different signaling pathways, depending on cellular localization. For example, active GSK-3 promotes cell spreading through phosphorylation of paxillin (14), and has been reported to both inhibit (15) and activate FAK (16). Local inhibition of GSK-3 at the leading edge of astrocytes promotes directed migration by regulating cell polarity (17).

GSK-3 regulates various aspects of cell motility including cytoskeletal dynamics, cell polarity and cell adhesion (4). Also, several GSK-3 substrates play a role in microtubule regulation including APC (17), CRMPs (18), and MAPs (19, 20). In addition to its direct effects on cytoskeletal organization, GSK-3 affects the activities of several transcription factors involved in regulation of cell proliferation and migration, including NF-κB, β-catenin and SNAIL (reviewed by (4)). The activities of these molecules, however, are as yet unknown in glioma migration and its role in glioma invasion has not been explored until the present invention herein.

The inventor herein now shows that pharmacologic inhibition of GSK-3 blocks glioma cell invasion in vitro, and that GSK-3β is active in migrating glioma cells both in vitro, and in a mouse glioma model. The inventor also shows that GSK-3 activity is essential for efficient glioma invasion, and therefore may represent a novel therapeutic target, TCF-lef luciferase reporter assay.

In another aspect, there is provided a method for developing therapeutic tools to prevent recurrence or further tumor spread and for developing compositions for affecting glioma cell dispersal comprising a GSK-3 inhibitor.

Also provided is a method for inhibiting brain tumor cell migration comprising administering an effective amount of a composition comprising one or more GSK-inhibitors.

In one particular aspect, there is provided herein a method for treating brain tumors, or for inhibiting or reducing symptoms of brain tumors in a patient. The method includes administering to the patient a therapeutically effective amount of a pharmaceutical composition which comprises a pharmaceutically acceptable amount of a GSK-3 specific inhibitor that is sufficient to block or inhibit activity of GSK-3 in the patient.

In certain embodiments, useful GSK-3 inhibitors or antagonists include Li+, SB415286 and AR-A014418, or a pharmaceutically acceptable salt or derivative thereof.

In certain other non-limiting embodiments, useful GSK-3 inhibitors or antagonists include bis-indole inhibitors such as indirubin compounds, including, for example, indirubin-3′ oxime, 6-bromoindirubin-3′ oxime, and 6-bromioindirubin-3′ acetoxime.

In certain other non-limiting embodiments, useful GSK-3 inhibitors or antagonists include of benzazepinone bis-indole inhibitors such as paullone compounds, including, for example, kenpaullone, alsterpaullone, and azakenpaullone.

In another broad aspect, the glial cell related disorder includes glial tumors. Non-limiting examples of glial tumors suitable for treatment include, but are not limited to, astrocytomas, glioblastomas, brain stem gliomas, ependymomas, oligodendrogliomas, optic nerve gliomas, subependymomas and mixed gliomas.

In another aspect, there is provided herein a pharmaceutical composition for treating glial tumors, or for inhibiting or reducing symptoms of glial tumors in a patient. The pharmaceutical composition can comprise a therapeutically effective amount of a pharmaceutically acceptable amount of a GSK-3 specific inhibitor that is sufficient to block or inhibit activity of GSK-3 in the patient, and a pharmaceutically acceptable excipient.

Also provided is a method for regenerating nerve cells comprising administering to a subject in need thereof, a therapeutically effective amount of a pharmaceutical composition which comprises a substance that inhibits the activity of GSK-3, as an active ingredient.

In one embodiment, the pharmaceutical composition comprising a GSK-3 inhibitor, or a pharmaceutically acceptable salt thereof, and a pharmaceutically acceptable carrier, at a therapeutically effective concentration, to prevent, inhibit or reverse glial tumors. In one aspect, GSK-3 inhibitor treatment is shown to inhibit growth of cultured tumor cells, as well as to inhibit tumor size in whole animals.

Administration of an “effective amount” or a “therapeutically effective amount” of a GSK-3 inhibitor means an amount that is useful, at dosages and for periods of time necessary to achieve the desired result. The therapeutically effective amount of a GSK-3 inhibitor may vary according to factors, such as the disease state, age, sex, and weight of the subject. Dosage regimens of a GSK-3 inhibitor, such as lithium, in the subject may be adjusted to provide the optimum therapeutic response. For example, several divided doses may be administered daily or the dose may be proportionally reduced as indicated by the exigencies of the therapeutic situation. In the context as used herein, a “pharmaceutically acceptable salt,” refer to salts prepared from pharmaceutically acceptable, non-toxic acids. Also, it is to be understood that pharmaceutical compositions can be present and administered in any suitable form.

The following examples are intended to illustrate preferred embodiments of the invention and should not be interpreted to limit the scope of the invention as defined in the claims.

### EXAMPLES

### Materials and Methods

Cell lines and chemicals. The U87 and U87ΔEGFR cell lines were provided by Dr. Webster Cavenee (Ludwig Institute for Cancer Research, La Jolla, Calif.). The human glioblastoma biopsies, X12 and X14 (from Dr. C. David James, Mayo Clinic, Rochester, Minn.), were maintained as flank tumors in nude mice as described (5). All cells were grown in DMEM with 10% FBS and 1% penicillin-streptomycin. Chemicals used were LiCl and myo-inositol (Sigma), AR-A014418 (Calbiochem), SB415286 (Tocris Bioscience) and L-690, 330 (Alexis Biochemicals), where SB415286 and AR-A0144418 are as follows:

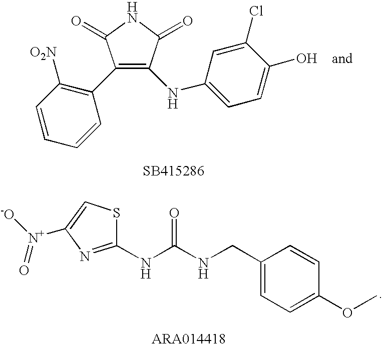

The β-catenin reporter plasmid pSuper8XTOPflash or pSuper8XFOPflash (control) (from Dr. Randall Moon, University of Washington, Wash.) (7) was transfected into U87 cells, seeded at 60,000 cells/well in a 12-well plate using Lipofectamine 2000. Promoter activity was determined by measuring luciferase levels in a Fluostar Optima plate reader (BMG Labtech).

In vivo studies.

Intracranial xenografts were performed using 6-week-old female nude mice (athymic nu/nu, NCI), according to Ohio State University animal safety regulations. 500,000 X14 cells were implanted 2 mm lateral and 1 mm anterior to the bregma, at a depth of 3 mm from the dura. After 25 days, brains were fixed with 4% paraformaldehyde in 0.9% NaCl and 10 mM sodium phosphate, pH 7.4 for 24 hours, followed by immersion in 30% sucrose in 10 mM sodium phosphate, pH 7.4, for 24 hours. 35 μm sections were mounted on gelatin-coated microscope slides.

Immunostaining and Western Blotting.

Sections were blocked with 1% rabbit serum in PBS and incubated overnight at 4° C. with rabbit anti-GSK-3β-[pS⁹] antibody (BioSource), followed by Hoechst 33258 and Alexa Fluor 488 conjugated secondary antibody (Molecular Probes), and mounted with Vectashield (Vector Laboratories). Negative controls were performed without primary antibodies. Actin staining of U87 cell spheroids in collagen I was performed with Alexa Fluor 568-phalloidin (Molecular Probes) according to manufacturers' recommendations. Brain slices were fixed in 4% paraformaldehyde, and stained with rabbit anti-vimentin antibody SP20 (Neomarkers, Fremont Calif.). For Western blotting, X12 tumors were excised from the flanks of nude mice, finely chopped and fractionated by successive filtration through 500 μm and 100 μm cell strainers. The aggregates were washed twice to remove any single cells, resuspended in neutralized collagen I solution and plated at approximately 200 aggregates per 10 cm² dish. Cells were harvested by treatment with 100 μg/ml type III collagenase (Sigma) in the presence of 0.36 mM CaCl₂ for 30 minutes at 37° C., followed by three washes with ice cold PBS. Cells were lysed in 50 mM Tris-HCl, pH 7.5, 100 mM NaCl, 1% TritonX100, 1 mM EDTA, 1 mM EGTA, 50 mM β-glycerophosphate, 1 mM DTT plus protease and phosphatase inhibitors. Proteins (30 μg) were separated by SDS-PAGE and transferred to nitrocellulose. Antibodies used for Western blotting were mouse anti-GSK-3β, mouse anti-GSK-3αβ mouse anti-β-catenin (BD biosciences), mouse anti-β-catenin-[pS^{33/37}, pT⁴¹] (Cell Signaling), mouse anti-α-tubulin (Sigma) and peroxidase conjugated secondary antibodies (Jackson Laboratories).

Results

Lithium Chloride Inhibits Glioblastoma Spheroid Invasion.

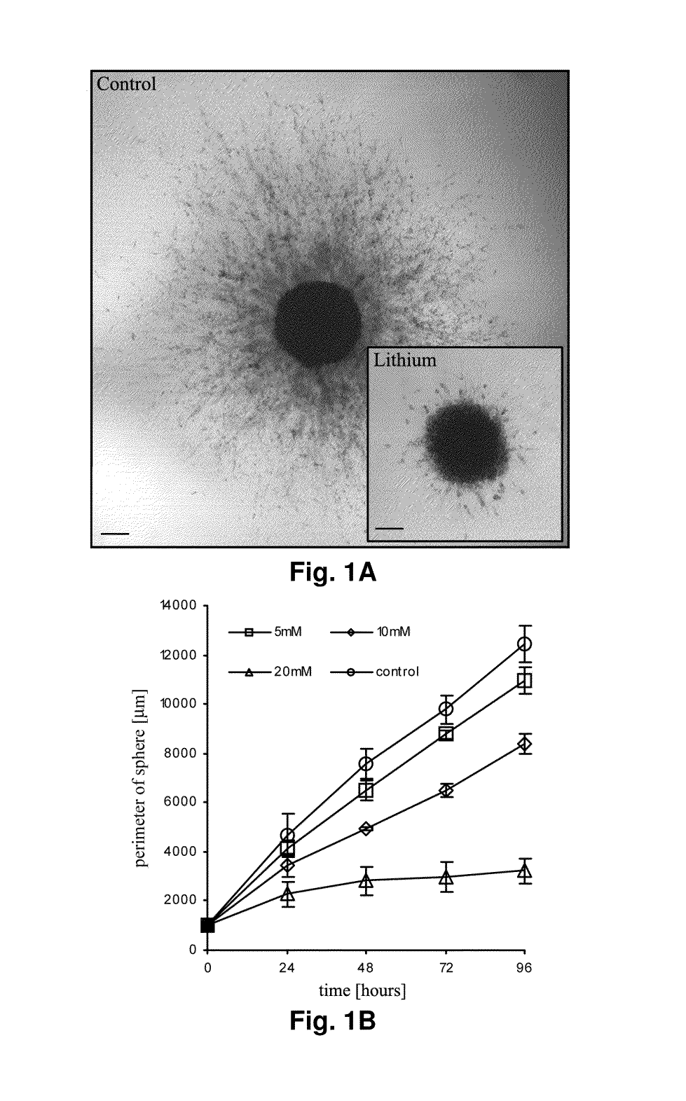

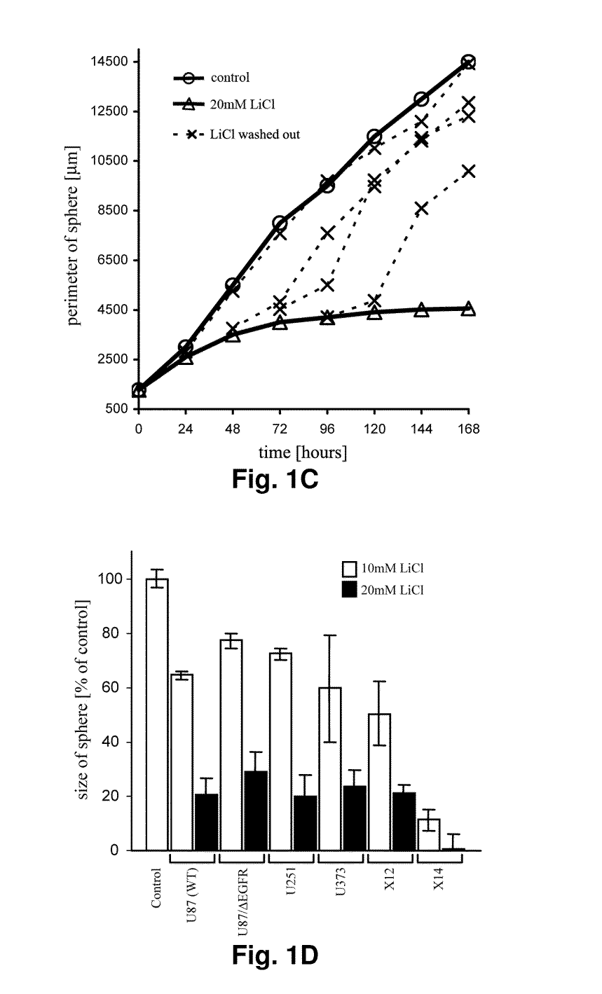

To investigate the role of GSK-3 in glioma invasion, experiments were carried out to examine the effects of lithium chloride (LiCl), a known inhibitor of GSK-3, on the invasion of U87 glioma cell spheroids embedded in a collagen I matrix. LiCl strikingly inhibited invasion of U87 glioma spheroids in a dose dependent manner, with maximum blockade at 20 mM LiCl. Under these conditions remarkably few cells are able to enter the matrix (FIGS. 1A and B).

The effect of 20 mM LiCl was reversible-cell migration resumed with similar kinetics to untreated spheres 24 hours after LiCl removal (FIG. 1C). This was observed for the duration of the assay (up to 96 hours), and demonstrated that inhibition of invasion is not due to irreversible cytotoxicity at 20 mM LiCl. Similar effects were observed with spheroids made from three additional glioma cell lines and two human glioblastoma biopsies maintained as flank tumors in nude mice (X12 and X14). Most had a similar dose-response, with X14 cells displaying the highest sensitivity (FIG. 1D). This may be because X14 cells migrated slowest in the spheroid assay.

Specific GSK-3 Inhibition Blocks Glioma Spheroid Invasion.

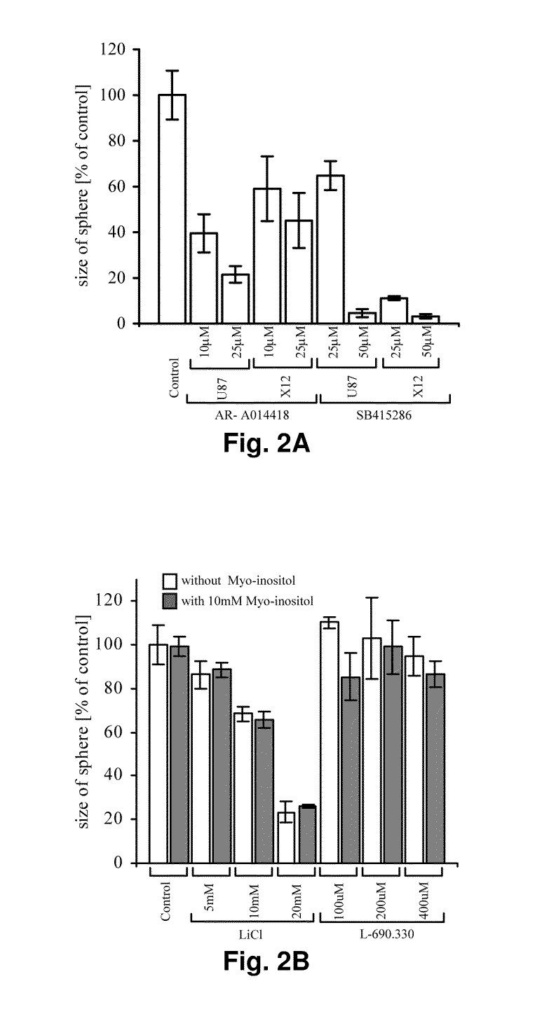

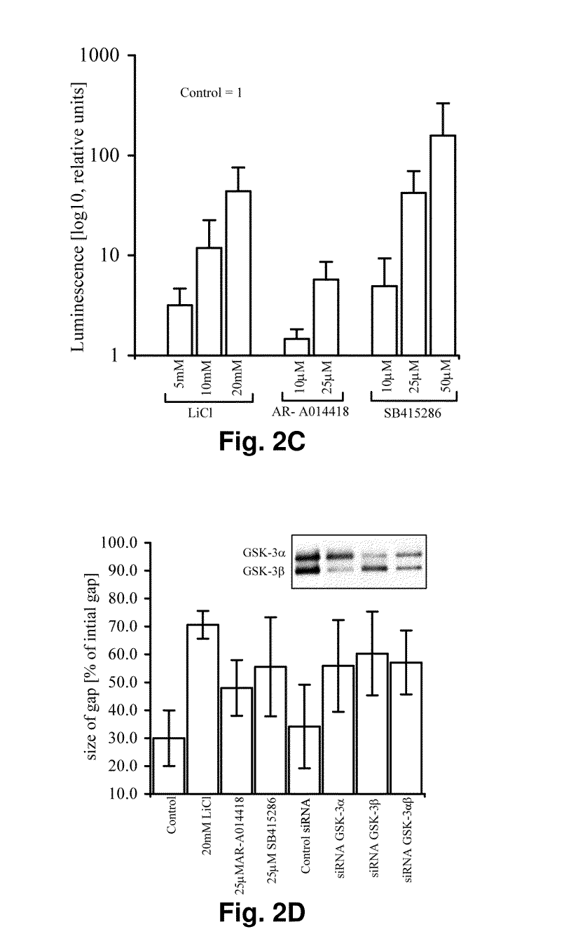

The best characterized molecular targets of lithium are GSK-3 and inositol monophosphatase (IMP). Experiments were carried out to examine the involvement of these molecules in glioma cell invasion/migration. First, two chemically distinct, potent and specific pharmacological GSK-3 inhibitors, SB4152 86 and AR-A014418, were examined in the spheroid invasion assay. Both inhibitors caused a dose-dependent blockade of invasion (FIG. 2A). This was observed in the full panel of cell lines and was reversible as described for LiCl. Minimal cytotoxicity was observed under conditions that block cell motility using propidium iodide exclusion and metabolic cell viability assays.

Blockade of IMP by lithium leads to depletion of cellular inositol levels, and certain effects of lithium can be rescued by addition of extracellular inositol (8). Excess inositol added to the spheroid assay did not rescue the LiCl blockade of invasion. In addition, L-690, 330, an IMP inhibitor 1000-fold more potent than lithium (9) had no effect on invasion even at concentrations as high as 400 μM (FIG. 2B). This data suggests that inositol metabolism is unlikely to be an important target of LiCl in the spheroid invasion model.

To further verify the role of GSK-3, the activity of a β-catenin responsive luciferase reporter plasmid (pSuper8XTOPflash) in the presence of GSK-3 inhibitors was analyzed in U87 cells. Inhibition of GSK-3 is known to correlate with increased transcription from the β-catenin responsive TCF/LEF binding sites in this vector due to β-catenin stabilization (7).

As shown herein, a dose-dependent increase of reporter activity was seen with all three inhibitors, consistent with GSK-3 inhibition (FIG. 2C). Immunostaining and Western blotting also showed an increase in GSK-3β serine-9 phosphorylation with inhibitor treatment (as has been previously reported due to pharmacological GSK-3 inhibition). To verify the effects of GSK-3 inhibition, wound-healing cell migration assays were performed. The assays showed a dose-dependent reduction in U373 glioma cell migration in the presence of GSK-3 inhibitors (FIG. 2D). To confirm the pharmacological data, siRNA was used to knockdown GSK-3α and GSK-3β expression. A 60% reduction was achieved in the expression of both isoforms, and this was reflected in reduced cell motility in wound-healing assays (FIG. 2D).

LiCl Treatment Breaks Down Long Extensions at the Leading Edge of Migrating Glioma Cells.

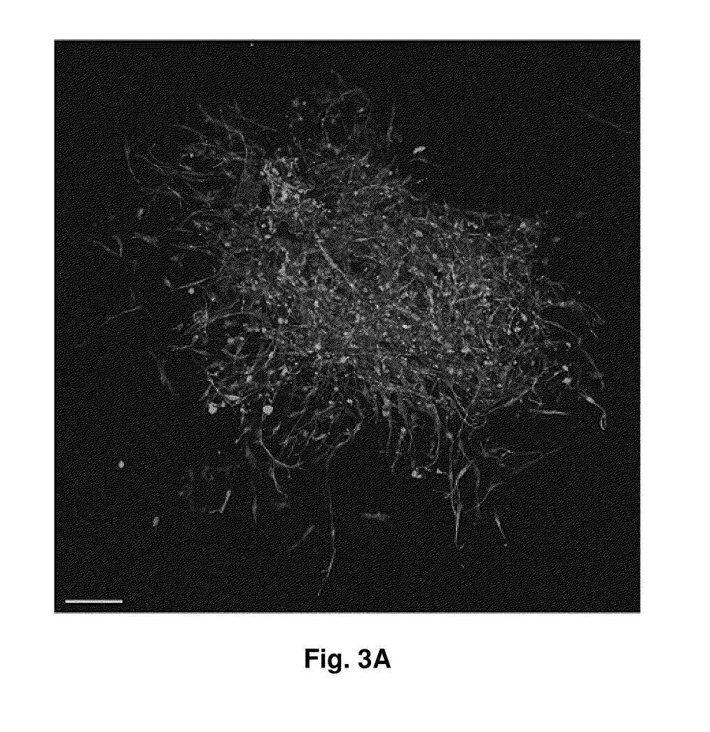

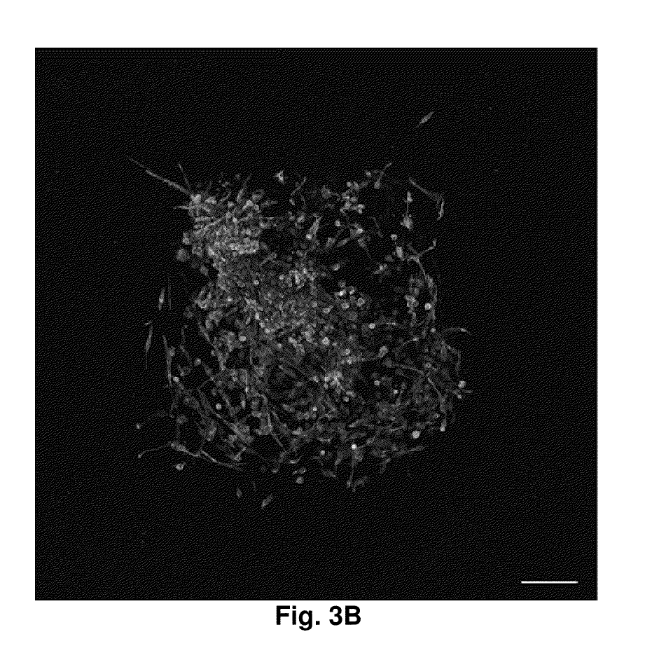

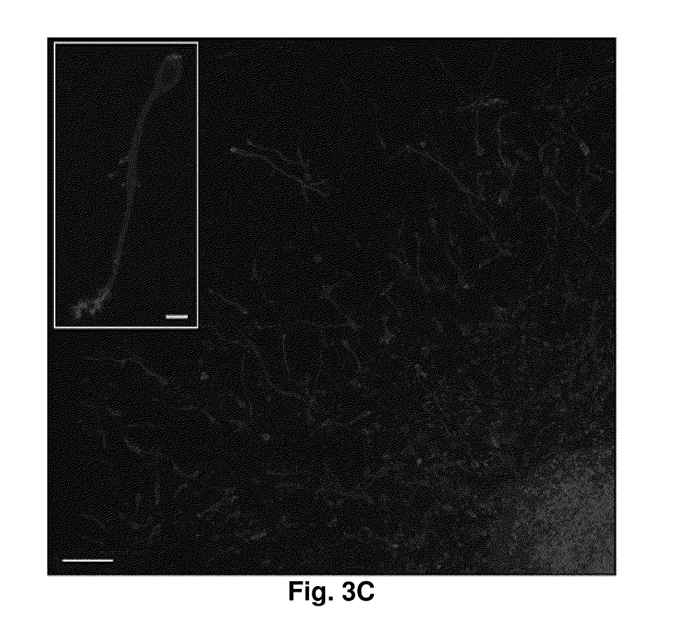

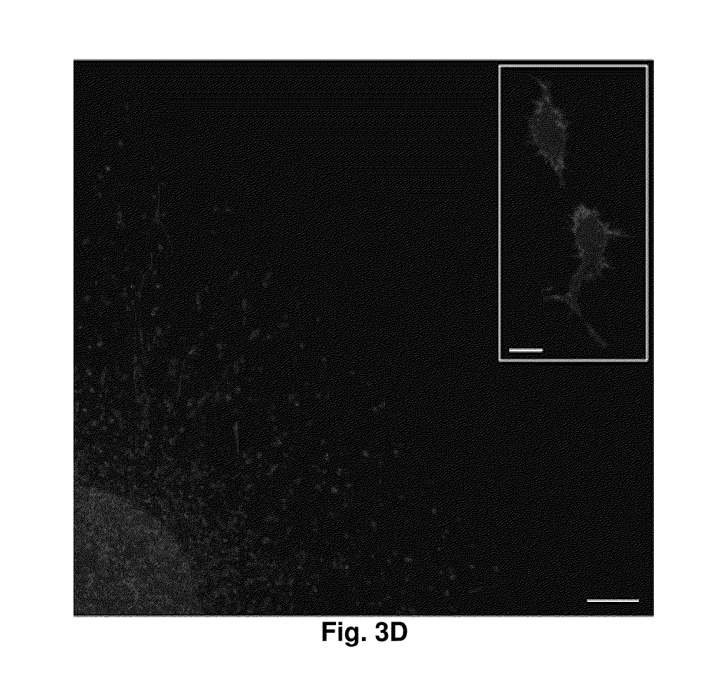

U87 glioma spheroids were cultured on brain slices prepared from newborn mice in order to examine glioma cell migration on a complex physiological matrix. Cells were able to migrate in this system, albeit slower than in type I collagen, and could be maintained for several weeks. Immersion of the brain slice in 20 mM LiCl for 24 hours slowed glioma cell migration and caused a marked change in shape, with becoming less elongated and rounding up (FIGS. 3A and B). This was also observed using X12 glioma cells. A similar change was seen when U87 glioma cells in the process of invading collagen I were incubated with 20 mM LiCl for 24 hours. This caused the cells to stop migrating 12 hours after drug addition, and was accompanied by the cells rounding up and retracting their long extensions (FIGS. 3C and D). Confocal microscopy using phalloidin to stain the actin cytoskeleton revealed that the long protrusions seen at the leading edge of migrating U87 cells had collapsed, although many actin rich filopodia were still observed.

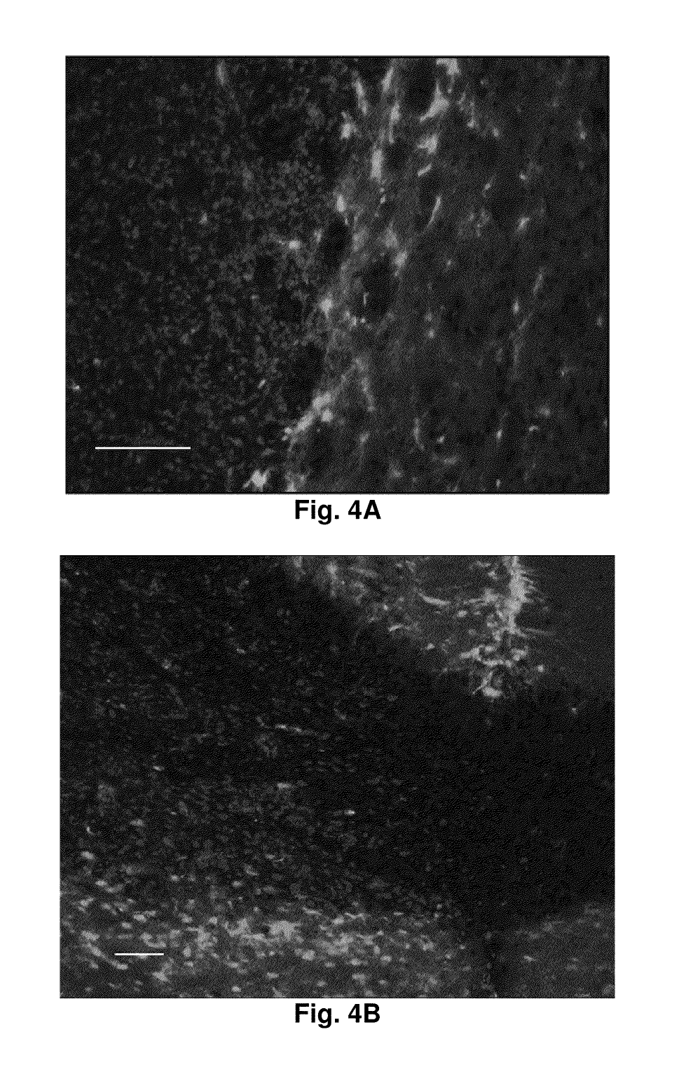

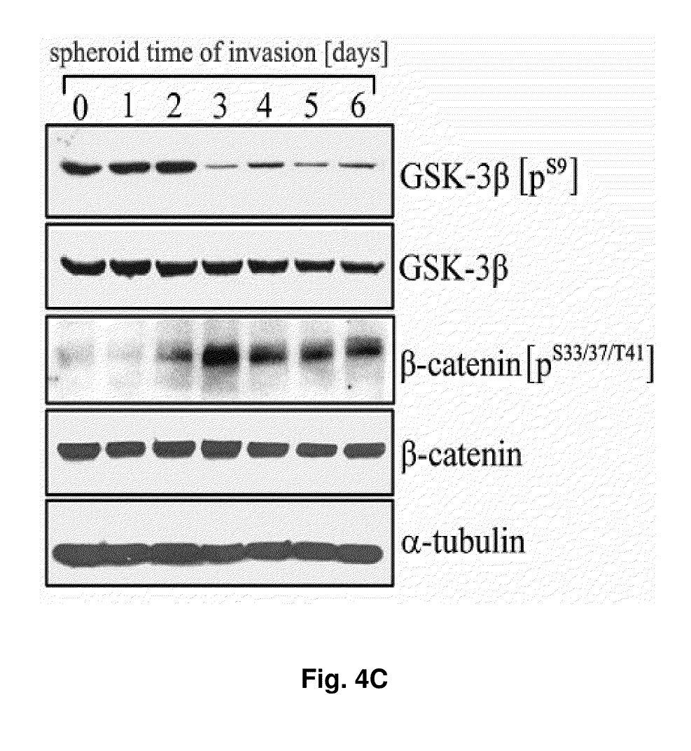

GSK-3 is catalytically active in migrating glioma cells. The blockade of invasion observed using GSK-3 inhibitors shows that the kinase activity of GSK-3 is required for efficient glioma invasion. Phospho-specific antibodies were used to examine the phosphorylation status of GSK-3 and one of its substrates, β-catenin, during glioma invasion. First, experiments were carried out in an in vivo model, in which X14 human glioma cells were implanted into the brain of a nude mouse. The catalytically inactive phospho-ser⁹ GSK-313 was readily detectable by immunostaining in normal brain, as compared with the tumor, suggesting GSK-313 is active glioma cells. This was clearly apparent in tumor cells invading the corpus collosum (FIG. 4B). Also, Western blotting of X12 glioma spheroids migrating in collagen in vitro revealed a marked decrease in phospho-ser⁹ GSK-3β during the course of migration, whereas overall GSK-313 levels were similar, providing further evidence that GSK-3 is active in these cells. In addition, phospho-β-catenin levels also increased during invasion, confirming that GSK-3β activity increases in invading cells.

Discussion

Pharmacologic GSK-3 inhibition potently blocks glioma cell invasion in a three-dimensional spheroid cell culture model. This system more faithfully reproduces the features of brain tumor invasion than monolayer cultures (process extension, matrix remodelling, adhesion to and detachment from the matrix) yet is a completely defined system, and easy to observe (as compared with in vivo models (6)).

The inventor herein shows that GSK-3 is an important mediator of glioma invasion. Firstly, three distinct small molecule GSK-3 inhibitors resulted in dose-dependent, reversible inhibition of glioma cell invasion in spheroid assays. Secondly, the degree of GSK-3 inhibition (measured by a luciferase reporter assay of β-catenin transcriptional activation), showed an inverse correlation with the degree of invasion. This now shows a direct link between GSK-3 activity and the rate of glioma invasion.

This is supported by the siRNA knockdown of either GSK-3α or GSK-3β which slowed migration in a wound healing assay, demonstrating that both GSK-3 isoforms play a role in glioma invasion. In addition, phospho-specific antibodies revealed i) decreased inhibitory phosphorylation of GSK-3β, and ii) increased phosphorylation of the GSK-3 substrate β-catenin in invading glioma cells. Cell migration was prevented with each inhibitor, in all glioma cell types examined, in spheroid, brain slice and wound healing assays. The brain slice model shows that GSK-3 inhibitors function in the context of a physiologically relevant matrix. It is also believed by the inventor herein that the mechanism is relevant in vivo, because the inhibitory phosphorylation of GSK-313 on serine 9 is barely detectable at the invasive tumor edge in a mouse xenograft model.

In another aspect, it is also shown herein shows that pharmacological inhibition of GSK-3 also disrupted cell polarity. This shows that either active GSK-3 is also required for cell polarity in an independent pathway, or that polarity requires dynamic GSK-3 regulation, with cycles of activation and inactivation, which would be prevented by GSK-3 inhibitors.

The breakdown of the long lamellipodia cellular structures in LiCl treated glioma cells shows that GSK-3 activity is important in regulating mechanisms that support these structures. Therefore, the inventor now believes that GSK-3 affects microtubule dynamics in glioma migration.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

The publication and other material used herein to illuminate the invention or provide additional details respecting the practice of the invention, are incorporated be reference herein, and for convenience are provided in the following bibliography.
1. Demuth, T. and Berens, M. E. Molecular mechanisms of glioma cell migration and invasion. J Neurooncol 2004; 70: 217-28.
2. Frame, S. and Cohen, P. GSK3 takes centre stage more than 20 years after its discovery. Biochem J 2001; 359: 1-16.
3. Doble, B. W. and Woodgett, J. R. GSK-3: tricks of the trade for a multi-tasking kinase. J Cell Sci 2003; 116: 1175-86.
4. Jope, R. S., Yuskaitis, C. J., and Beurel, E. Glycogen Synthase Kinase-3 (GSK3): Inflammation, Diseases, and Therapeutics. Neurochem Res 2006.
5. Giannini, C., Sarkaria, J. N., Saito, A. et al. Patient tumor EGFR and PDGFRA gene amplifications retained in an invasive intracranial xenograft model of glioblastoma multiforme. Neuro-oncol 2005; 7: 164-76.
6. Del Duca, D., Werbowetski, T., and Del Maestro, R. F. Spheroid preparation from hanging drops: characterization of a model of brain tumor invasion. J Neurooncol 2004; 67: 295-303.
7. Veeman, M. T., Axelrod, J. D., and Moon, R. T. A second canon. Functions and mechanisms of beta-catenin-independent Wnt signaling. Dev Cell 2003; 5: 367-77.
8. Harwood, A. J. Lithium and bipolar mood disorder: the inositol-depletion hypothesis revisited. Mol Psychiatry 2005; 10: 117-26.
9. Phiel, C. J. and Klein, P. S. Molecular targets of lithium action. Annu Rev Pharmacol Toxicol 2001; 41: 789-813.
10. Farooqui, A. A., Ong, W. Y., and Horrocks, L. A. Inhibitors of brain phospholipase A2 activity: their neuropharmacological effects and therapeutic importance for the treatment of neurologic disorders. Pharmacol Rev 2006; 58: 591-620.
11. Koivisto, L., Hakkinen, L., Matsumoto, K. et al. Glycogen synthase kinase-3 regulates cytoskeleton and translocation of Rac1 in long cellular extensions of human keratinocytes. Exp Cell Res 2004; 293: 68-80.
12. Owen, R. and Gordon-Weeks, P. R. Inhibition of glycogen synthase kinase 3beta in sensory neurons in culture alters filopodia dynamics and microtubule distribution in growth cones. Mol Cell Neurosci 2003; 23: 626-37.
13. Lefranc, F., Yeaton, P., Brotchi, J., and Kiss, R. Cimetidine, an unexpected anti-tumor agent, and its potential for the treatment of glioblastoma (review). Int J Oncol 2006; 28: 1021-30.
14. Cai, X., Li, M., Vrana, J., and Schaller, M. D. Glycogen synthase kinase 3- and extracellular signal-regulated kinase-dependent phosphorylation of paxillin regulates cytoskeletal rearrangement. Mol Cell Biol 2006; 26: 2857-68.
15. Bianchi, M., De Lucchini, S., Marin, O. et al. Regulation of FAK Ser-722 phosphorylation and kinase activity by GSK3 and PP1 during cell spreading and migration. Biochem J 2005; 391: 359-70.
16. Kobayashi, T., Hino, S., Oue, N. et al. Glycogen synthase kinase 3 and h-prune regulate cell migration by modulating focal adhesions. Mol Cell Biol 2006; 26: 898-911.
17. Etienne-Manneville, S, and Hall, A. Cdc42 regulates GSK-3beta and adenomatous polyposis coli to control cell polarity. Nature 2003; 421: 753-6.
18. Yoshimura, T., Kawano, Y., Arimura, N. et al. GSK-3beta regulates phosphorylation of CRMP-2 and neuronal polarity. Cell 2005; 120: 137-49.
19. Sanchez, C., Perez, M., and Avila, J. GSK3beta-mediated phosphorylation of the microtubule-associated protein 2C (MAP2C) prevents microtubule bundling. Eur J Cell Biol 2000; 79: 252-60.
20. Goold, R. G., Owen, R., and Gordon-Weeks, P. R. Glycogen synthase kinase 3beta phosphorylation of microtubule-associated protein 1B regulates the stability of microtubules in growth cones. J Cell Sci 1999; 112 (Pt 19): 3373-84.

What is claimed is:

1. A method of treating a glial cell related disorder in a mammalian subject comprising administering a drug to the subject, wherein the drug enhances or prolongs GSK-3 α or β inactivation.

2. The method of claim 1, wherein the drug can block glial cell invasion or inhibit glial cell migration.

3. The method of claim 1, wherein the glial cell related disorder comprises one or more gliomas.

4. The method of claim 1, wherein the glial cell related disorder leads to glioma spheroid expansion.

5. The method of claim 1, wherein the subject is in need or regenerating nerve cells.

6. The method of claim 1, wherein the drug comprises one or more of: LiCl, SB415286 and AR-A-14418.

7. The method of claim 1, wherein the glial cell related disorder is characterized by misregulation of GSK-3.

8. The method of claim 1, wherein the glial cell related disorder comprises inhibiting recurrence of a glioma tumor.

9. A method for screening or identifying a compound useful in treating glial cell related disorders, comprising identifying inhibitory and stimulatory compounds capable of enhancing or prolonging GSK-3 α or β inactivation.

10. The method of claim 1 useful as a therapeutic tool to prevent recurrence or further tumor spread.

11. A medicament for treating a glial cell related disorder in a mammalian subject comprising administering a drug to the subject, wherein the medicament comprises a drug which drug enhances or prolongs GSK-3 α or β inactivation.

12. The medicament of claim 11, wherein the drug can block glial cell invasion or inhibit glial cell migration.

13. The medicament of claim 11, wherein, wherein the glial cell related disorder comprises one or more gliomas.

14. The medicament of claim 11, wherein, wherein the glial cell related disorder leads to glioma spheroid expansion.

15. The medicament of claim 11, wherein, wherein the subject is in need or regenerating nerve cells.

16. The medicament of claim 11, wherein, wherein the drug comprises one or more of: LiCl, SB415286 and AR-A-14418.

17. The medicament of claim 11, wherein, wherein the glial cell related disorder is characterized by misregulation of GSK-3.

18. The medicament of claim 11, wherein, wherein the glial cell related disorder comprises inhibiting recurrence of a glioma tumor.

19. A biomarker for glioma cell invasion or migration comprising glycogen synthase kinase-3 (GSK-3).

20. A method for evaluating invasion or migration of glial cells comprising using glycogen synthase kinase-3 (GSK-3) as a biomarker therefor.
